(19) United States
(12) Patent Application Publication
(10) Pub. No.: US 2008/0131955 A1
(43) Pub. Date: Jun. 5, 2008

(54) Method of Separating Target DNA from Mixed DNA

(75) Inventor: Michele R. Stone, Rockville, MD (US)

Correspondence Address: ROTHWELL, FIGG, ERNST & MANBECK, P.C., 1425 K STREET, N.W., SUITE 800, WASHINGTON, DC, 20005, US

(73) Assignee: CANON U.S. LIFE SCIENCES, INC., Rockville, MD (US)

(21) Appl. No.: 11/946,538

(22) Filed: Nov. 28, 2007

Related U.S. Application Data
(60) Provisional application No. 60/884,232, filed on Jan. 10, 2007.
(60) Provisional application No. 60/867,939, filed on Nov. 30, 2006.

(51) Int. Cl. C12N 1/08

(52) U.S. Cl. 435/270

(57) ABSTRACT

The present invention relates to methods of separating target DNA from mixed DNA in a sample. In some embodiments, the target DNA is present in target organisms. In other embodiments, the target organisms may be viruses, bacteria, fungi or combinations thereof. In some embodiments the sample contains cells having nuclei. In some embodiments the cells are mammalian cells. In some embodiments, the separated target organisms are treated to release their DNA which can be recovered.

This application claims the benefit of Provisional Patent Application No. 60/884,232, filed on Jan. 10, 2007 and Provisional Patent Application No. 60/867,939, filed on Nov. 30, 2006, each of which is incorporated herein by reference.

### BACKGROUND

1. Field of the Invention

The present invention relates to methods of separating target DNA from mixed DNA in a sample. In some embodiments, the target DNA is present in target organisms. In other embodiments, the target organisms may be viruses, bacteria, fungi or combinations thereof. In some embodiments the sample contains cells having nuclei. In some embodiments the cells are mammalian cells. In some embodiments, the separated target organisms are treated to release their DNA which can be recovered.

2. Description of Related Art

The detection of nucleic acids is central to medicine. The ability to detect infectious organisms (e.g., viruses, bacteria, fungi) is ubiquitous technology for disease diagnosis and prognosis. Determination of the integrity of a nucleic acid of interest can be relevant to the pathology of an infection. One of the most powerful and basic technologies to detect small quantities of nucleic acids is to replicate some or all of a nucleic acid sequence many times, and then analyze the amplification products. PCR is perhaps the most well-known of a number of different amplification techniques. The nucleic acids are generally isolated from a sample prior to detection, although in situ detection can also be performed.

The basic steps of nucleic acid, such as DNA, isolation are disruption of the cellular structure to create a lysate, separation of the soluble nucleic acid from cell debris and other insoluble material, and purification of the DNA of interest from soluble proteins and other nucleic acids. Historically, organic extraction (e.g., phenol:chloroform) followed by ethanol precipitation was done to isolate DNA. Disruption of most cells is done by chaotropic salts, detergents or alkaline denaturation, and the resulting lysate is cleared by centrifugation, filtration or magnetic clearing. The DNA can then be purified from the soluble portion of the lysate. When silica matrices are used, the DNA is eluted in an aqueous buffer such as Tris-EDTA (TE) or nuclease-free water.

DNA isolation systems for genomic, plasmid and PCR product purification are historically based on purification by silica. Regardless of the method used to create a cleared lysate, the DNA of interest can be isolated by virtue of its ability to bind silica in the presence of high concentrations of chaotropic salts (Chen and Thomas, Anal Biochem 101:339-341, 1980; Marko et al., Anal Biochem 121:382-387, 1982; Boom et al., J Clin Microbiol 28:495-503, 1990). These salts are then removed with an alcohol-based wash and the DNA eluted in a low ionic strength solution such as TE buffer or water. The binding of DNA to silica seems to be driven by dehydration and hydrogen bond formation, which competes against weak electrostatic repulsion (Melzak et al., J Colloid and Interface Science 181:635-644, 1996). Hence, a high concentration of salt will help drive DNA adsorption onto silica, and a low concentration will release the DNA.

Recently, new methods for DNA purification have been developed which take advantage of the negatively charged backbone of DNA to a positively charged solid substrate (under specific pH conditions), and eluting the DNA using a change in solvent pH (ChargeSwitch® technology, Invitrogen, Corp., Carlsbad, Calif.; see, for example, U.S. Pat. No. 6,914,137 and International Published Application No. 2006/004611). Whatman has an alternate technology (FTA® paper) that utilizes a cellulose based solid substrate impregnated with a lysis material that lyses cells, inactivates proteins, but captures DNA in the cellulose fibers, where it is retained for use in downstream applications (see, for example, U.S. Pat. No. 6,322,983). Regardless of the applications there is no way to use any of the above described technologies to separate viral, bacterial or fungal DNA from mixed DNA.

There are technologies currently available that utilize fractioning cells into subcellular organelles and compartments. This is beneficial in studying the functions of these compartments as well as the macromolecules housed in the compartments. The study of the nucleus and nuclear events is one of the largest areas of research currently being conducted. The ability to understand a number of processes of primary importance in cell biology, including chromatin structure, transcriptional regulation of gene expression, RNA synthesis and processing, mechanism of regulation of bi-directional nuclear transport and nuclear apoptosis, are critical in understanding most of the diseases that plague mammals in general.

There are several commercial kits that offer buffers to lyse cells while keeping the nucleus intact. Sigma offers the Nuclei EZ Prep Nuclei Isolation Kit (NUC-100) and Bio Vision has the Nuclear/Cytosol Fractionation Kit (K266-100). Many kits, however, have an intermediate step of isolating nuclei, in the purification of RNA and nuclear specific proteins.

In kits that are commercially available a hypotonic buffer is provided that will lyse the outer membrane of a mammalian cell. This liberates the cytosolic components; however, the nucleus remains intact. With the nucleus intact the mammalian DNA can be separated from the other cellular components by centrifugation. The function of the nuclei isolation kits is to enrich for either mammalian DNA, RNA, or for nuclear proteins, for the further study. However, they are not used to remove mammalian DNA from a mixed DNA sample.

United States Patent Application Publication No. 2005/10142663 mentions prior art for isolating white blood cells or their nuclei by centrifugation after treatment with a hypotonic lysis buffer. This reference also discusses using this method to enrich mammalian DNA in a microfluidic system.

Early detection of infectious agents in a mammalian tissue sample, such as whole blood, requires that a few infectious agent DNA molecules be detected in a background of many mammalian tissue DNA molecules. Separation of the infectious agent DNA molecules from the mammalian tissue DNA molecules would improve detection efficiencies by lowering the background of mammalian DNA in the sample. None of the above described methods address the problem of purifying bacterial, viral, or fungal DNA separately from mixed DNA, including mammalian DNA, in a mixed DNA sample. Thus, a need exists for methods that provide for the enrichment and purification of viral, bacterial or fungal DNA in the presence of mixed DNA.

### SUMMARY OF THE INVENTION

The present invention relates to methods of separating target DNA from mixed DNA in a sample. In some embodiments, the target DNA is present in target organisms. In some embodiments, the target organisms may be viruses, bacteria (prokaryotes), fungi or combinations thereof. In some embodiments the mixed DNA includes target DNA and non-target DNA. In some embodiments, the non-target DNA is mammalian DNA. In some embodiments the sample contains cells having nuclei. In some embodiments the cells are mammalian cells. In some embodiments, the separated target organisms are treated to release their DNA which can be recovered.

Thus, the present invention provides a method of separating target DNA from mixed DNA in a cellular sample comprising: (a) selectively lysing cells in the cellular sample and (b) filtering the selectively lysed sample to separate the target DNA from the non-target DNA. In some embodiments, the cells contain target organisms containing target DNA and nuclei containing non-target DNA. In other embodiments, the target organisms are selected from the group consisting of viruses, bacteria, fungi and combinations thereof. In a further embodiment, the selective lysis involves lysing the outer membranes of the cells while only partially lysing the nuclear membranes of the cells such that the nucleus remains intact. The selective lysis of the cell membrane occurs with or without lysing the target organisms. In some embodiments, the non-target DNA is mammalian DNA. In other embodiments, the cellular sample is a blood sample, a urine sample, a saliva sample, a sputum sample, a cerebrospinal fluid sample, a body fluid sample or a tissue sample. In some embodiments, the selective lysis is performed by contacting the cellular sample with a buffer that selectively permeabilizes cellular membranes while leaving the nuclei of the cells intact. In other embodiments the buffer that selectively permeabilizes cellular membranes is a hypotonic buffer. In additional embodiments, the buffer that selectively permeabilizes cellular membranes is an isotonic buffer. In other embodiments, the buffer further comprises a detergent. In additional embodiments, the detergent is an ionic detergent or a non-ionic detergent. In some embodiments, the filtering is performed using a 5 μm or less filter. In some embodiments, the filter is less than 5 μm. In other embodiments, the filter is 4 μm or less. In additional embodiments, the filter is 3 μm or less. In further embodiments, the filter is 2.7 μm. In some embodiments, the method further comprises collecting the filtrate containing the target organisms. In other embodiments, the method further comprises lysing the target organism to release the target DNA. In further embodiments, the method further comprises recovering the target DNA for downstream applications.

In a second aspect, the present invention provides a method of separating target DNA from mammalian DNA in a cellular sample comprising: (a) selectively lysing cells in the cellular sample and (b) filtering the selectively lysed sample to separate the target DNA from the mammalian DNA. In some embodiments, the cells are mammalian cells that contain target organisms containing target DNA and nuclei containing mammalian DNA. In other embodiments, the target organisms are selected from the group consisting of viruses, bacteria, fungi and combinations thereof. In other embodiments, the target DNA is selected from the group consisting of viral DNA, bacterial DNA, fungal DNA and combinations thereof. In a further embodiment, the selective lysis involves lysing the outer membranes of the cells without lysing the nuclear membranes of the cells and without lysing the target organisms. In some embodiments, the non-target DNA is mammalian DNA. In other embodiments, the cellular sample is a blood sample, a urine sample, a saliva sample, a sputum sample, a cerebrospinal fluid sample, a body fluid sample or a tissue sample. In some embodiments, the selective lysis is performed by contacting the cellular sample with a buffer that selectively permeabilizes cellular membranes while leaving the nuclei of the cells intact. In other embodiments the buffer that selectively permeabilizes cellular membranes is a hypotonic buffer. In additional embodiments, the buffer that selectively permeabilizes cellular membranes is an isotonic buffer. In other embodiments, the buffer further comprises a detergent. In additional embodiments, the detergent is an ionic detergent or a non-ionic detergent. In some embodiments, the filtering is performed using a 5 μm or less filter. In some embodiments, the filter is less than 5 μm. In other embodiments, the filter is 4 μm or less. In additional embodiments, the filter is 3 μm or less. In further embodiments, the filter is 2.7 μm. In some embodiments, the method further comprises collecting the filtrate containing the target organisms. In other embodiments, the method further comprises lysing the target organism to release the target DNA. In further embodiments, the method further comprises recovering the target DNA for downstream applications.

The above and other embodiments of the present invention are described below with reference to the accompanying drawings.

### BRIEF DESCRIPTION OF THE DRAWINGS

The accompanying drawings, which are incorporated herein and form part of the specification, illustrate various embodiments of the present invention. In the drawings, like reference numbers indicate identical or functionally similar elements.

FIG. 1 illustrates a mammalian cell containing a nucleus which contains the mammalian DNA and also containing bacteria.

FIG. 2 illustrates selective separation of target organisms containing target DNA from nucleic containing mammalian DNA.

FIG. 3 shows quantitative PCR results for the selective removal of mammalian DNA in a mixed DNA sample.

### DETAILED DESCRIPTION OF THE INVENTION

The present invention has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, Oligonucleotide Synthesis: A Practical Approach, 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

As described above, there are no methods which address the problem of purifying bacterial, viral, and/or fungal DNA separately from mammalian DNA in a mixed DNA sample. The present invention provides for the enrichment and purification of bacterial, viral and/or fungal DNA in the presence of mammalian DNA. Thus, the present invention relates to methods for separating target DNA from non-target DNA in a mixed DNA sample.

The present invention provides for the separation of non-target DNA, e.g., mammalian DNA, from target DNA, e.g., bacterial, viral and/or fungal DNA, by utilizing the unique characteristic of DNA packaging within the target organisms and non-target organisms or cells and the unique properties of cellular and nuclear membranes. Mammalian DNA is found within the nucleus of a cell which has a typical size of 5-7 μm. Target DNA is found within a virus, viral particle, bacterium or fungal cell, all of which have typical sizes well below the size of a nucleus. In addition, cellular and nuclear membranes have different properties which allow for selective permeabilization of cellular membranes. The present invention takes advantage of the different packaging and different membrane properties to provide for the enrichment for bacterial, viral and/or fungal DNA over mammalian DNA.

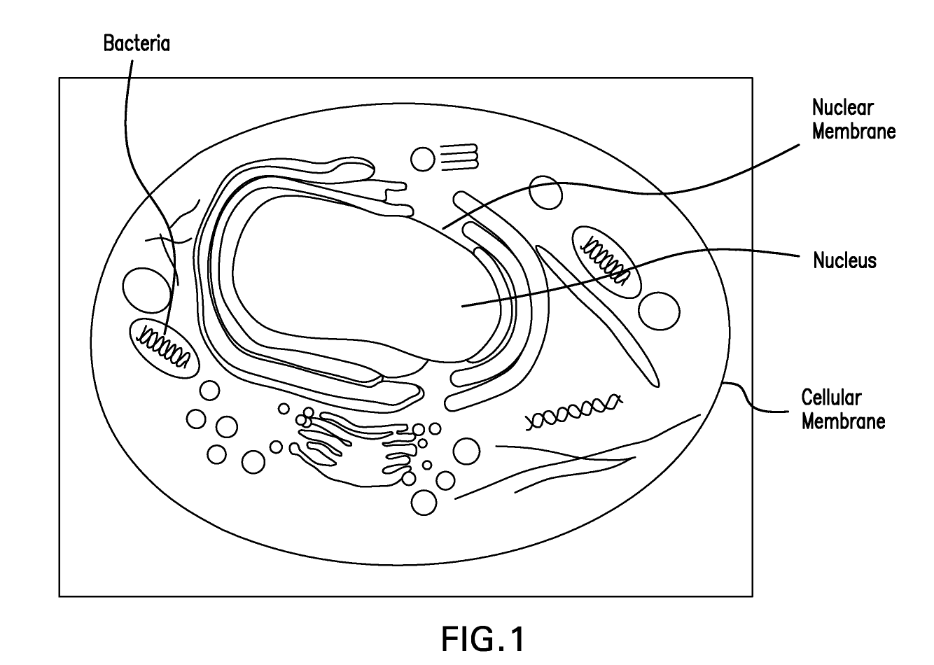

The packaging of target DNA and mammalian DNA is illustrated in FIG. 1 which illustrates a mammalian cell with bacteria. The cell has a large nucleus along with various other cellular components. The nucleus contains the non-target DNA. This cell has either been invaded by bacteria or has engulfed bacteria from the external medium. The size difference between the nucleus and the bacteria is significant. The bacteria are typically 1-3 μm while the nuclei range from 5-7 μm.

Thus, in a first aspect, the present invention provides a method of separating target DNA from mixed DNA in a cellular sample comprising: (a) selectively lysing cells in the cellular sample and (b) filtering the selectively lysed sample to separate the target DNA from the non-target DNA. In some embodiments, the cells contain target organisms containing target DNA and nuclei containing non-target DNA. In other embodiments, the cellular sample contains target organisms containing target DNA. In some embodiments, the target organisms are selected from the group consisting of viruses, bacteria, fungi and combinations thereof. In some embodiments, the non-target DNA is mammalian DNA.

The selective lysis involves lysing the outer membranes of the cells without lysing the nuclear membranes of the cells and without lysing the target organisms present in the cells or the cellular sample. The cellular sample may be a blood sample, a urine sample, a saliva sample, a sputum sample, a cerebrospinal fluid sample, a body fluid sample or a tissue sample. The selective lysis is performed by contacting the cellular sample with a buffer that selectively permeabilizes cellular membranes while leaving the nuclei of the cells intact. This same buffer also leaves the target organisms intact. Buffers having these properties are well known to the skilled artisan. Products that include buffers for selectively lysing cellular membranes are commercially available. Suitable commercial products that include such buffers, include, but are not limited to, Nuclei EZ Prep Nuclei Isolation Kit (NUC-101) (Sigma, St. Louis, Mo., USA), Nuclear/Cytosol Fractionation Kit (K266-100) (BioVision Research Products, Mountain View, Calif., USA), NE-PER Nuclear and Cytoplasmic Extraction Reagents (Pierce, Rockville, Ill., USA), Nuclear Extraction Kit (Imgenex, Corp., San Diego, Calif., USA), Nuclear Extract Kit (Active Motif, Carlsbad, Calif., USA), and Qproteome Nuclear Protein Kit (Qiagen, Valencia, Calif., USA). See also, U.S. Pat. Nos. 5,447,864, 6,852,851 and 7,262,283. It is well known that the type of nuclei in question will determine which buffer will be required. See, U.S. Pat. No. 5,447,864 for a discussion of factors that can be optimized for preparing a suitable selective lysis buffer for different cell types.

In one embodiment, the buffer is a hypotonic buffer. For example, commercial hypotonic lysis buffer can be purchased from Sigma Aldrich, Nuclei EZ lysis buffer (N 3408). A kit is also available from Sigma Aldrich, Nuclei EZ Prep Nuclei Isolation Kit (Nuc-101). A common recipe for a 10× hypotonic solution is, 100 mM HEPES, pH 7.9, with 15 mM MgCl₂ and 100 mM KCl. In another embodiment, the buffer is a hypotonic buffer that comprises a detergent. Suitable detergents include, but are not limited to ionic detergents, such as lithium lauryl sulfate, sodium deoxycholate, and Chaps, or non-ionic detergents, such as Triton X-100, Tween 20, Np-40, and IGEPAL CA-630. In another embodiment, the buffer is an isotonic buffer. For example, Sigma Aldrich offers a kit, CelLytic Nuclear Extraction kit, which contains an isotonic lysis buffer. A common recipe for a 5× isotonic lysis buffer is, 50 mM Tris HCl, pH 7.5, with 10 mM MgCl₂, 15 mM CaCl₂, and 1.5M Sucrose. In an additional embodiment, the buffer is an isotonic buffer that comprises a detergent which may be an ionic detergent or a non-ionic detergent.

In some embodiments, the filtering is performed using a 5 μm or less filter. In some embodiments, the filter is less than 5 μm. In other embodiments, the filter is 4 μm or less. In additional embodiments, the filter is 3 μm or less. In further embodiments, the filter is 2.7 μm. Conventional filters having the desired size cutoffs are well known to the skilled artisan.

In some embodiments, the method further comprises collecting the filtrate containing the target organisms. In other embodiments, the method further comprises lysing the target organisms to release the target DNA. Conventional techniques to lyse the target organisms are used and are well known to the skilled artisan. Suitable techniques include, but are not limited to, the use of detergents, alkali, heat, physical disruption (e.g., mechanical, homogenization, acoustic energy and pressure), and the like. Products that contain reagents for the lysis of bacteria, viruses and fungi are also commercially available. Suitable commercial products include, but are not limited to, Poppers Cell Lysis Reagents (Pierce, Rockville, Ill., USA), chemagic DNA Bacteria Kit (Parallabs, Inc., Worcester, Mass., USA), Bactozol™ Kit Bacterial DNA Isolation Kit (Molecular Research Center, Inc., Cincinnati, Ohio, USA), QIAamp DNA Mini Kit (Qiagen, Valencia, Calif., USA), ChargeSwitch® gDNA Mini Bacteria Kit (Invitrogen, Corp, Carlsbad, Calif., USA), and MasterPure™ Yeast DNA Purification Kit (Epicentre Biotechnologies, Madison, Wis., USA) In further embodiments, the method further comprises recovering the target DNA for downstream applications. The recovered target DNA can be concentrated prior to use in downstream applications. Many of the previously listed kits also include DNA recovery and isolation following lysis of the viruses, bacteria and/or fungi.

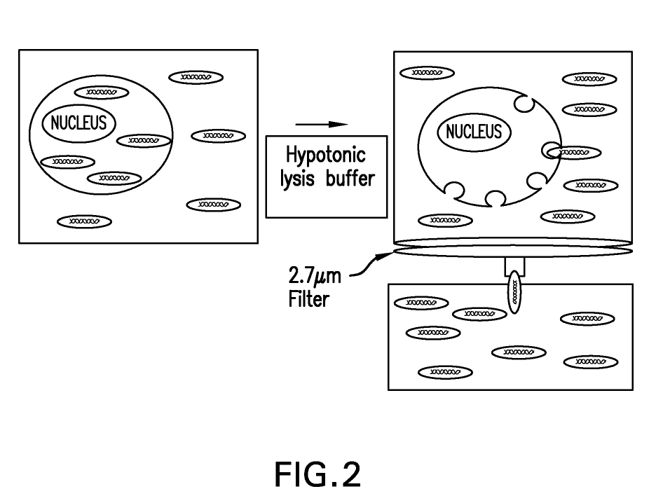

FIG. 2 illustrates selective removal of mammalian DNA from bacterial or viral DNA. This figure depicts the selective lysis of the cellular membrane and release of cytosolic components, while the nucleus remains intact. The mammalian DNA can be removed from the sample by passing the medium through a 2.7 μm filter, and collecting the filtrate. The filtrate will be enriched for bacterial and viral DNA while the mammalian DNA is removed by the filter. The filtrate can be treated to release the target DNA which can be recovered for downstream processing as described herein.

Experimental results indicate that this method is 90-99% efficient at removing mammalian DNA from a mixed DNA sample, while enriching for target DNA. In this experiment, Salmonella at 10⁶ copies was serially diluted in blood to a final dilution of 10² copies. Hypotonic lysis buffer (Nuclei EZ lysis buffer (N 3408) from Sigma Aldrich) was added and samples were incubated 4 minutes at room temperature. Samples were passed through a 2.7 μm filter, filtrates were collected and DNA was purified by using the Precision System Science USA's (Livermore, Calif.) Magtration® 12 GC automated DNA purification system. Total DNA extracted from filtrates was analyzed using QPCR assays specific for nuclear DNA and for bacterial target DNA, fluorescence spectroscopy using Pico Green dye, and gel electrophoresis. To confirm that nuclei remained intact during the cell lysis procedure, lysates were stained prior to filtration with two fluorescent DNA binding dyes, Hoechst 3342 which is membrane permeable, and propidium iodide which is membrane impermeable.

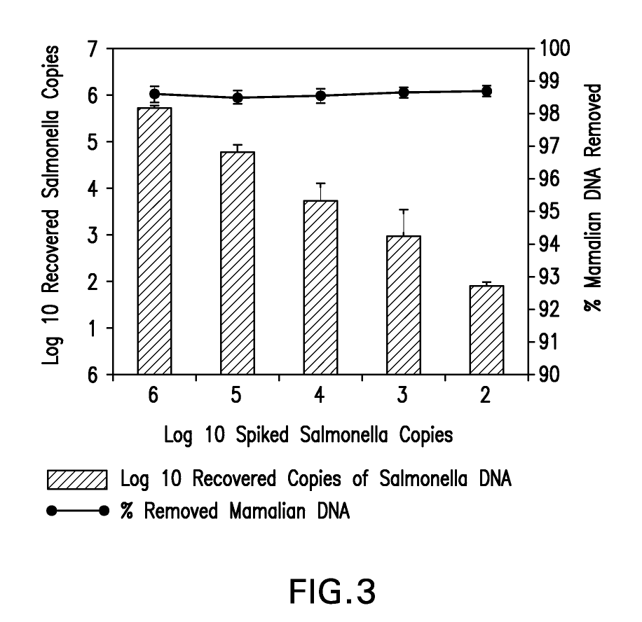

FIG. 3 shows quantitative PCR (qPCR) results for the selective removal of mammalian DNA in a mixed DNA sample. The data represent a minimum of 2 samples run in duplicate. Each sample was run in duplicate in a qPCR reaction to quantitate the removed mammalian DNA and the recovered bacterial DNA. The dual fluorescent nuclear staining procedure showed that the efficiency of cell lysis in the hypotonic lysis buffer was 97%. The Pico Green assay results showed that 70% (SD=11.5%) of the total DNA was removed with filtration technique. The qPCR results showed that 98.6% (SD=0.12%) of the mammalian genomic DNA was removed, while 66% (SD=12%) of the bacterial DNA was recovered. Agarose gel analysis of the DNA extracts showed no visible genomic DNA band in the filtered samples and clearly visible bands in the non-filtered samples. These results show that there is a 98-99% removal of mammalian DNA from bacterial DNA and that there is a significant recovery of bacterial targets even at low copy numbers. With the mammalian DNA largely removed from the sample, it is much easier to concentrate the remaining DNA and achieve lower LOD in molecular diagnostic assays.

In a second aspect, the present invention provides a method of separating target DNA from mammalian DNA in a cellular sample comprising: (a) selectively lysing mammalian cells in the cellular sample and (b) filtering the selectively lysed sample to separate the target DNA from the mammalian DNA. In some embodiments, the mammalian cells contain target organisms containing target DNA. In other embodiments, the target organisms are selected from the group consisting of viruses, bacteria, fungi and combinations thereof. In other embodiments, the target DNA is selected from the group consisting of viral DNA, bacterial DNA, fungal DNA and combinations thereof.

The selective lysis involves lysing the outer membranes of the cells without lysing the nuclear membranes of the cells and without lysing the target organisms present in the cells or the cellular sample as described herein. The cellular sample may be a blood sample, a urine sample, a saliva sample, a sputum sample, a cerebrospinal fluid sample, a body fluid sample or a tissue sample. The selective lysis is performed by contacting the cellular sample with a buffer that selectively permeabilizes cellular membranes while leaving the nuclei of the cells intact. This same buffer also leaves the target organisms intact. Buffers having these properties are well known to the skilled artisan and are as described herein. In some embodiments, the filtering is performed using a 5 μm or less filter, e.g. less than 5 μm or 4 μm or less or 3 μm or less or 2.7 μm as described herein. In some embodiments, the method further comprises collecting the filtrate containing the target organisms. In other embodiments, the method further comprises lysing the target organism to release the target DNA as described herein. In further embodiments, the method further comprises recovering the target DNA as described herein for downstream applications. The recovered target DNA can be concentrated prior to use in downstream applications.

The present invention allows for the selective removal of mammalian DNA from a mixed sample. The present invention uses a filter to isolate the nuclei and separate it from the sample, while previous methods use centrifugation to collect nuclei. The present invention method can be performed at room temperature while previous published methods and kits require the use of ice cold buffer and the storage of samples on ice throughout the purification process. Overall, the present method is extremely efficient at removing mammalian DNA from cellular samples while allowing for the enrichment of bacterial, viral and/or fungal DNA.

The current state of the art in molecular diagnostics for infectious disease does not include separation of bacterial, viral and/or fungal DNA from background mammalian DNA in tissue extracts. Instead the mixed sample is utilized for the specific amplification and detection of the target bacterial, viral and/or fungal DNA. In many cases the background mammalian DNA interferes with amplification and detection. The present invention can be used to remove background mammalian DNA prior to the amplification and detection steps of diagnostic procedures for the bacterial, viral and/or fungal DNA. The present method is an extremely powerful tool in the selective removal of mammalian DNA in a mixed DNA blood sample.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of separating target DNA from mixed DNA in a cellular sample comprising:
(a) selectively lysing cells in the cellular sample, wherein the cells contain target DNA and nuclei containing non-target DNA, wherein the target DNA is selected from the group consisting of viral DNA, bacterial DNA, fungal DNA and combinations thereof, wherein the target DNA is in target organisms and wherein outer membranes of the cells are lysed without lysing nuclear membranes of the cells; and
(b) filtering the selectively lysed sample to separate the target DNA from the non-target DNA.

2. The method of claim 1, wherein the target DNA is present in the cells in a virus or a viral particle.

3. The method of claim 1, wherein the target DNA is present in the cells in a bacterium.

4. The method of claim 1, wherein the target DNA is present in the cells in a fungal cell.

5. The method of claim 1, wherein the non-target DNA is mammalian DNA.

6. The method of claim 1, wherein the cellular sample is selected from the group consisting of a blood sample, a urine sample, a saliva sample, a sputum sample, a cerebrospinal fluid sample, a body fluid sample and a tissue sample.

7. The method of claim 1, wherein the selective lysis is performed by contacting the cellular sample with a buffer that selectively permeabilizes cellular membranes and leaves the nuclear membranes intact.

8. The method of claim 7, wherein the buffer is a hypotonic buffer.

9. The method of claim 8, wherein the buffer comprises a detergent.

10. The method of claim 9, wherein the detergent is an ionic detergent or a non-ionic detergent.

11. The method of claim 7, wherein the buffer is an isotonic buffer.

12. The method of claim 11, wherein the buffer comprises a detergent.

13. The method of claim 12, wherein the detergent is an ionic detergent or a non-ionic detergent.

14. The method of claim 1, wherein the filtering is performed using a 5 μm or less filter.

15. The method of claim 14, wherein the filter is less than 5 μm.

16. The method of claim 15, wherein the filter is 4 μm or less.

17. The method of claim 15, wherein the filter is 3 μm or less.

18. The method of claim 15, wherein the filter is 2.7 μm.

19. The method of claim 1 which further comprises collecting the filtrate containing the target organisms.

20. The method of claim 19 which further comprises lysing the target organisms to release the target DNA and recovering the target DNA.

21. A method of separating target DNA from mammalian DNA in a cellular sample comprising:
(a) selectively lysing cells in the cellular sample, wherein the cells are mammalian cells containing target DNA and nuclei containing mammalian DNA, wherein the target DNA is selected from the group consisting of viral DNA, bacterial DNA, fungal DNA and combinations thereof, wherein the target DNA is in target organisms and wherein outer membranes of the cells are lysed without lysing nuclear membranes of the cells; and
(b) filtering the selectively lysed sample to separate the target DNA from the mammalian DNA.

22. The method of claim 21, wherein the target DNA is present in the cells in a virus or a viral particle.

23. The method of claim 21, wherein the target DNA is present in the cells in a bacterium.

24. The method of claim 21, wherein the target DNA is present in the cells in a fungal cell.

25. The method of claim 21, wherein the cellular sample is selected from the group consisting of a blood sample, a urine sample, a saliva sample, a sputum sample, a cerebrospinal fluid sample, a body fluid sample and a tissue sample.

26. The method of claim 21, wherein the selective lysis is performed by contacting the cellular sample with a buffer that selectively permeabilizes cellular membranes and leaves the nuclear membranes intact.

27. The method of claim 26, wherein the buffer is a hypotonic buffer.

28. The method of claim 27, wherein the buffer comprises a detergent.

29. The method of claim 28, wherein the detergent is an ionic detergent or a non-ionic detergent.

30. The method of claim 26, wherein the buffer is an isotonic buffer.

31. The method of claim 30, wherein the buffer comprises a detergent.

32. The method of claim 31, wherein the detergent is an ionic detergent or a non-ionic detergent.

33. The method of claim 21, wherein the filtering is performed using a 5 μm or less filter.

34. The method of claim 33, wherein the filter is less than 5 μm.

35. The method of claim 34, wherein the filter is 4 μm or less.

36. The method of claim 34, wherein the filter is 3 μm or less.

37. The method of claim 34, wherein the filter is 2.7 μm.

38. The method of claim 21 which further comprises collecting the filtrate containing the target organisms.

39. The method of claim 38 which further lysing the target organisms to release the target DNA and recovering the target DNA.
